(19) United States
(12) Patent Application Publication
(10) Pub. No.: US 2011/0245799 A1
(43) Pub. Date: Oct. 6, 2011

(54) SYSTEM AND METHOD FOR DELIVERY OF SUSPENSIONS AND OTHER MICROPARTICLE COMPOSITIONS

(76) Inventors: SIGNE R. ERICKSON, Menlo Park, CA (US); Charles J. Hagemeier, Laguna Beach, CA (US); Gary A. Winchester, Warrior, AL (US)

(21) Appl. No.: 13/077,291

(22) Filed: Mar. 31, 2011

Related U.S. Application Data
(60) Provisional application No. 61/426,775, filed on Dec. 23, 2010.
(60) Provisional application No. 61/319,762, filed on Mar. 31, 2010.

(51) Int. Cl. A61M 5/31; A61M 5/315

(52) U.S. Cl. 604/506; 604/218; 604/92

(57) ABSTRACT

Disclosed herein are systems and methods useful for delivery of suspensions and other microparticle compositions, and, in particular, for delivering microparticles at desired dosing levels and eliminating blockages within microparticle suspensions positioned within a delivery device.

### CROSS-REFERENCE TO RELATED APPLICATIONS

This application claims the benefit of priority to U.S. Provisional Patent Application Ser. No. 61/319,762, filed on Mar. 31, 2010, and U.S. Provisional Patent Application Ser. No. 61/426,775, filed on Dec. 23, 2010, the entire disclosures of which are incorporated by reference herein for all purposes.

### FIELD

Disclosed herein are systems and methods for delivery of suspensions and other microparticle compositions. More particularly, the disclosed systems and methods can be used to deliver microparticle compositions, including suspensions, at desired therapeutic dosing levels to a targeted tissue and, optionally, to eliminate blockages within a microparticle composition positioned within a delivery device.

### BACKGROUND

Most drugs in development and approved for treating “back of the eye” diseases are injected directly into the vitreous humor, a thick clear gel that fills the space between the lens and retina. To date, the focus of the injection technique has centered around prevention of infection, and little work has been done with respect to the location and formulation of the injected material. The importance in controlling the dosage level and distribution of injected materials in the eye has become particularly apparent when delivering microparticle formulations. Without controlling the injection procedure and other formulation variables, these microparticles can float into the visual field over time, or adhere to other ocular tissues. To address the safety and efficacy of these systems, more control over initial dosing levels and distribution is needed. Injection techniques, surgical instrumentation, and formulation variables all play roles in controlling the initial location of injected material in the eye. These factors have been refined herein to limit the migration and distribution of injected material over time.

Additionally, when the injected material is a suspension, the injected material can typically only be injected into a subject using large-gauge needles, such as 19 and 20 gauge (or larger) needles. However, injections accomplished using these large-gauge needles can cause significantly more pain than injections accomplished using smaller-gauge needles, such as 23 gauge (and smaller) needles. For example, in order to minimize pain in subjects, intramuscular injections are typically performed using 23-26 gauge needles. Although smaller-gauge needles help reduce the pain experienced by subjects, these needles typically cannot be used to inject suspensions. As the size of the needle decreases, there is an increased likelihood that blockages of the needle will occur during injection of a suspension.

Thus, there is a need in the pertinent art for systems and methods for maintaining efficacious levels of therapeutic material proximal to the disease site while preventing adverse effects, such as obstruction of the visual field and interaction with and damage to the retina and lens. Additionally, there is a need in the pertinent art for systems and methods for reducing the pain associated with injections of therapeutic suspensions. More specifically, there is a need in the pertinent art for systems and methods for eliminating blockages that occur during injections of therapeutic suspensions, particularly in injections accomplished using smaller-gauge needles.

### SUMMARY

Disclosed herein are systems and methods useful for delivery of a selected therapeutic composition, such as a suspension or other microparticle composition. For ease in understanding, portions of the disclosure discuss the systems and methods being used in conjunction with ocular administration. However, this is not meant to be limiting, as it is contemplated that the methods and systems described herein can be used for other applications and in other desired tissues in which control of the dosing level and distribution of the injected materials is desired.

Additional advantages will be set forth in part in the description that follows, and in part will be obvious from the description, or can be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below.

FIG. 1 is a schematic illustration of one embodiment of the system for delivery of a therapeutic material, showing a cartridge coupled to a sonic energy-generating means, as described herein.

FIG. 2 is a schematic illustration of one embodiment of a system for delivery of a therapeutic material, showing a housing that encloses a cartridge that is coupled to a sonic energy-generating means, as described herein.

FIG. 3 is a schematic illustration of one embodiment of the system for delivery of a therapeutic material, showing a first cartridge coupled to a second cartridge, and showing a vibrational collar and a sonic energy-generating means operatively coupled to the second cartridge.

FIG. 4 is a schematic illustration of one embodiment of the system for delivery of a therapeutic material, showing a first cartridge coupled to a second cartridge via a connector.

### DETAILED DESCRIPTION

The present invention can be understood more readily by reference to the following detailed description, examples, and claims, and their previous and following description. However, before the present compositions, articles, devices, and/or methods are disclosed and described, it is to be understood that this invention is not limited to the specific compositions, articles, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its currently known embodiments. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

In this specification and in the claims that follow, reference will be made to a number of terms that shall be defined to have the following meanings:

Throughout this specification, unless the context requires otherwise, the word “comprise,” or variations such as “comprises” or “comprising,” will be understood to imply the inclusion of a stated integer or step or group of integers or steps but not the exclusion of any other integer or step or group of integers or steps.

It must be noted that, as used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, a “wt. %” or “weight percent” or “percent by weight” of a component, unless specifically stated to the contrary, refers to the ratio of the weight of the component to the total weight of the composition in which the component is included, expressed as a percentage.

As used herein, “contacting” means the physical contact of at least one substance with at least one other substance.

As used herein, “sufficient amount” and “sufficient time” means an amount and time needed to achieve the desired result or results, e.g., dissolve a portion of the polymer.

“Admixture” or “blend” as generally used herein means a physical combination of two or more different components. In the case of polymers, an admixture, or blend, of polymers is a physical blend or combination of two or more different polymers as opposed to a copolymer which is single polymeric material that is comprised of two or more different monomers.

“Molecular weight” as used herein, unless otherwise specified, refers generally to the relative average molecular weight of the bulk polymer. In practice, molecular weight can be estimated or characterized in various ways including gel permeation chromatography (GPC) or capillary viscometry. GPC molecular weights are reported as the weight-average molecular weight (Mw) or as the number-average molecular weight (Mn). Capillary viscometry provides estimates of molecular weight as the Inherent Viscosity (IV) determined from a dilute polymer solution using a particular set of concentration, temperature, and solvent conditions. Unless otherwise specified, IV measurements are made at 30° C. on solutions prepared in chloroform at a polymer concentration of 0.5 g/dL.

“Controlled release” as used herein means the use of a material to regulate the release of another substance.

“Excipient” is used herein to include any other compound or additive that can be contained in or on the microparticle that is not a therapeutically or biologically active compound. As such, an excipient should be pharmaceutically or biologically acceptable or relevant (for example, an excipient should generally be non-toxic to the subject). “Excipient” includes a single such compound and is also intended to include a plurality of excipients.

“Agent” is used herein to refer generally to compounds that are contained in or on a microparticle composition. Agent can include a bioactive agent or an excipient. “Agent” includes a single such compound and is also intended to include a plurality of such compounds “Biocompatible” as used herein refers to a material that is generally non-toxic to the recipient and does not possess any significant untoward effects to the subject and, further, that any metabolites or degradation products of the material are non-toxic to the subject.

“Biodegradable” is generally referred to herein as a material that will erode to soluble species or that will degrade under physiologic conditions to smaller units or chemical species that are, themselves, non-toxic (biocompatible) to the subject and capable of being metabolized, eliminated, or excreted by the subject.

The term “microparticle” is used herein to include nanoparticles, microspheres, nanospheres, microcapsules, nanocapsules, and particles, in general. As such, the term microparticle refers to particles having a variety of internal structure and organizations including matrices such as microspheres (and nano spheres) or core-shell matrices (such as microcapsules and nanocapsules), porous particles, multi-layer particles, among others. The term “microparticle” refers generally to particles that have sizes in the range of about 10 nanometers (nm) to about 2 mm (millimeters).

“Subject” is used herein to refer to any target of administration. The subject can be a vertebrate, for example, a mammal. Thus, the subject can be a human. The term does not denote a particular age or sex. Thus, adult and newborn subjects, as well as fetuses, whether male or female, are intended to be covered. A “patient” refers to a subject afflicted with a disease or disorder and includes human and veterinary subjects.

As used herein, the “elimination” of blockages as described herein refers to any removal, dispersal, or break-up of a blockage or clog occurring within a cartridge that hinders flow of a composition from within the cartridge to a needle or other element in fluid communication with the cartridge.

Disclosed are compounds, compositions, and components that can be used for, can be used in conjunction with, and can be used in preparation for, the disclosed systems and methods. These and other materials are disclosed herein, and it is understood that when combinations, subsets, interactions, groups, etc. of these materials are disclosed that while specific reference of each various individual and collective combination and permutation of these compounds may not be explicitly disclosed, each is specifically contemplated and described herein. For example, if a number of different polymers and agents are disclosed and discussed, each and every combination and permutation of the polymer and agent are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

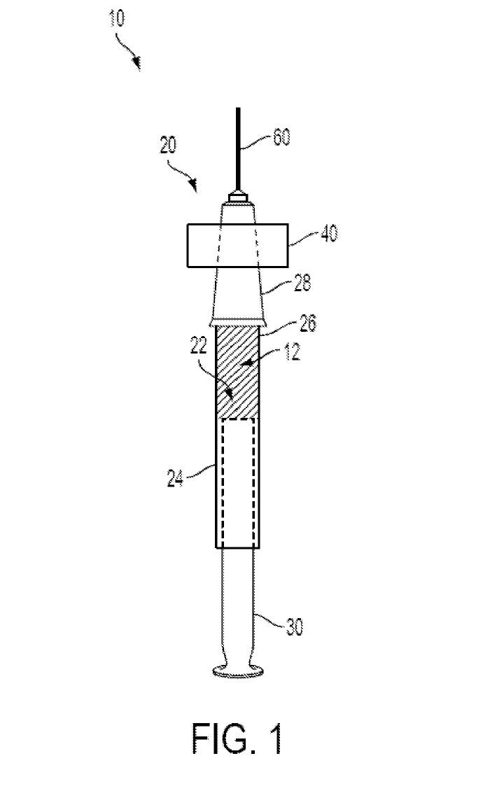

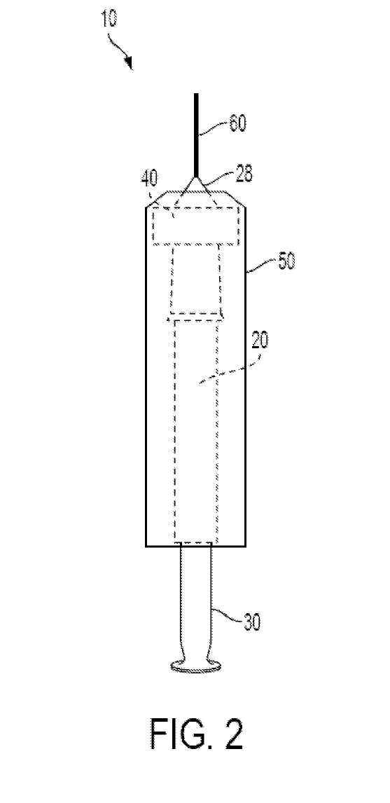

In a broad aspect of the invention, and as depicted in FIGS. 1-2, a system 10 for delivery of a selected composition 12, such as a suspension, is provided, the system generally comprising a needle 60, a cartridge 20 configured for fluid-tight connection to the needle and having an internal cavity 22 containing a predetermined amount of the selected composition an axially movable plunger 30 positioned within the internal cavity of the cartridge, and a sonic energy-generating means 40 for contacting a portion of the cartridge. In a further aspect, the system 10 is configured to allow selective activation of the sonic energy-generating means 40 during movement of the axially moveable plunger 30. In this aspect, it is contemplated that the sonic energy-generating means 40 can be configured to eliminate blockages within the internal cavity 22 of the cartridge 20. It is further contemplated that the sonic energy-generating means 40 can be selectively activated to enable a medical practitioner to use smaller needles than would conventionally be used to inject the selected composition into a subject, thereby reducing the level of pain experienced by the subject. In exemplary non-limiting aspects, the selected composition 12 can be a therapeutic composition. For example, in one aspect, the selected composition 12 can be a suspension comprising microparticles. However, it is contemplated that the selected composition 12 can be any therapeutic composition having desired properties for particular applications and at particular injection sites within a subject.

In one aspect, the needle 60 can have a desired size for insertion into selected tissue of a subject. It is contemplated that the gauge of the needle 60 can be minimized as appropriate to reduce pain in the subject following injection of the selected composition 12 into the selected tissue of the subject. It is further contemplated that the use of a needle or other device having a body member cross section with a reduced diameter can limit the number of sutures required to close tissue of a subject following completion of an injection or other procedure. For example, it is contemplated that, following implantation of one or more compositions into an eye of a subject using a needle having a minimal diameter, few or no sutures will be required to accomplish scleral closure in the eye of the subject. In one non-limiting example, the needle 60 can have a gauge ranging from 24 G to 30 G, including 24 G, 25 G, 26 G, 27 G, 28 G, 29 G, and 30 G. However, it is contemplated that the needle 60 can have any gauge that is suitable for a particular injection into the subject.

In another aspect, and with reference to FIG. 1, the predetermined amount of the selected composition 12 can be sealed therein the internal cavity 22 of the cartridge 20. In additional aspects, the cartridge 20 can have an outer surface 24 and a distal end 26. In one aspect, the distal end 26 of the cartridge 20 can be configured for fluid-tight connection to the needle 60. In this aspect, the distal end 26 of the cartridge 20 can have a luer-lock configuration 28. In exemplary non-limiting aspects, the axially moveable plunger 30 can be positioned in a friction fit within the internal cavity 22 of the cartridge 20.

In a further aspect, the sonic energy-generating means 40 can be configured to contact a portion of the outer surface 24 of the cartridge 20. In one exemplary non-limiting aspect, the sonic energy-generating means 40 can be configured to contact the distal end 26 of the cartridge 20. For example, in this aspect, the sonic energy-generating means 40 can be configured to contact a portion of a luer-lock configuration 28 of the cartridge 20, such as, for example and without limitation, a hub of the luer-lock configuration. It is contemplated that the sonic energy-generating means 40 can be configured to eliminate any blockages existing proximate the interface between the cartridge 20 and the needle 60 to thereby promote flow of the selected composition from the cartridge and into the needle. In still a further aspect, the sonic energy-generating means 40 can be mounted thereto the outer surface 24 of the cartridge 20. In this aspect, as shown in FIG. 1, the sonic energy-generating means 40 can comprise a collar element positioned around a portion of the cartridge 20. In another aspect, the sonic energy-generating means 40 can comprise a sonic energy probe, such as, for example and without limitation: digital probe models S-150, 250, and 450 manufactured by Branson Sonifier; 250 and 400 Watt Sonic Ruptor probes manufactured by Omni International; and a model 3000 ultrasonic homogenizer manufactured by Biologics, Inc.

In an additional aspect, the sonic energy-generating means 40 can have a desired power output. In this aspect, it is contemplated that the desired power output of the sonic energy-generating means 40 can range from about 1 to about 140 Watts. In another aspect, it is contemplated that the desired power output of the sonic energy-generating means 40 can range from about 10 to about 120 Watts. In still another aspect, it is contemplated that the desired power output of the sonic energy-generating means 40 can range from about 20 to about 50 Watts. Thus, in various aspects, it is contemplated that the desired power output of the sonic energy-generating means 40 can be 1, 10, 20, 30, 40, 50, 60, 70, 80, 90, 100, 110, 120, 130, or 140 Watts. It is further contemplated that the desired power output of the sonic energy-generating means 40 can fall within a range derived from any two of the above-listed values. Similarly, it is contemplated that the desired power output of the sonic energy-generating means 40 can be any power output falling between any two of the above-listed values.

In a further aspect, the sonic energy-generating means 40 can be in operative communication with a power source. In one exemplary aspect, the power source can be a rechargeable battery. However, it is contemplated that any other conventional power source can be used to activate the sonic energy-generating means 40.

In another aspect, and with reference to FIG. 2, the system 10 can comprise a housing 50. In this aspect, it is contemplated that the needle 60, the cartridge 20, and the axially-moveable plunger 30 can be secured within at least a portion of the housing 50. In an additional aspect, the sonic energy-generating means 40 can be incorporated into the housing 50 such that the sonic energy-generating means is positioned proximate the distal end 26 of the cartridge 20. Alternatively, the sonic energy-generating means 40 can be secured within the housing 50 such that the sonic energy-generating means is positioned proximate the distal end 26 of the cartridge 20. In one aspect, the housing 50 can be configured to receive the power source of the sonic energy-generating means 40. In another aspect, the power source of the sonic energy-generating means 40 can be positioned external to the housing 50. In this aspect, it is contemplated that the power source can be mounted thereto an outer portion of the housing 50. In a further aspect, the power source of the sonic energy-generating means 40 can be incorporated into the housing 50.

It is contemplated that movement of the axially-moveable plunger 30 and activation of the sonic energy-generating means 40 can be selectively controllable by a user from outside the housing 50. For example, in one aspect, the system 10 can comprise a controller including an on-off mechanism, such as, for example and without limitation, an on-off switch and an on-off button, that is selectively moveable between an on position and an off position, wherein the on position corresponds to activation of the sonic energy-generating means 40. In this aspect, it is contemplated that the controller can be positioned thereon the housing 50. It is further contemplated that the controller can comprise means for adjustably controlling the power output of the sonic energy-generating means 40. In a further aspect, it is further contemplated that a proximal portion of the axially moveable plunger 30 can be accessible by the user from outside the housing 50.

In another exemplary aspect, the housing 50 can be shaped to conform to the shape of a user's hand. For example and without limitation, it is contemplated that the housing 50 can be substantially cylindrical. It is further contemplated that the housing 50 can have a substantially pen-like shape. In one aspect, the housing 50 can comprise at least one of an injectable moldable plastic and stainless steel. It is contemplated that the housing 50 can comprise an inner surface and an outer surface that are easily cleanable with conventional cleaning materials.

In operation, it is contemplated that the selected composition can be delivered into a subject by: sealing a predetermined amount of the selected composition into the internal cavity of a cartridge as described herein; selectively axially moving a plunger positioned in a friction fit within the internal cavity of the cartridge; contacting at least a portion of the outer surface of the cartridge with the sonic energy-generating means; and selectively activating the sonic energy-generating means to eliminate blockages within the internal cavity of the cartridge. In one aspect, the step of contacting at least a portion of the outer surface of the cartridge with the sonic energy-generating means can comprise contacting the distal end of the cartridge with the sonic energy-generating means. In another aspect, it is contemplated that the step of selectively activating the sonic energy-generating means can comprise selectively generating sonic energy at a power output ranging from about 1 Watt to about 140 Watts. In yet another aspect, it is contemplated that the step of selectively activating the sonic energy-generating means can comprise selectively generating sonic energy at a power output ranging from about 10 Watts to about 120 Watts. In still another aspect, it is contemplated that the step of selectively activating the sonic energy-generating means can comprise selectively generating sonic energy at a power output ranging from about 20 Watts to about 50 Watts.

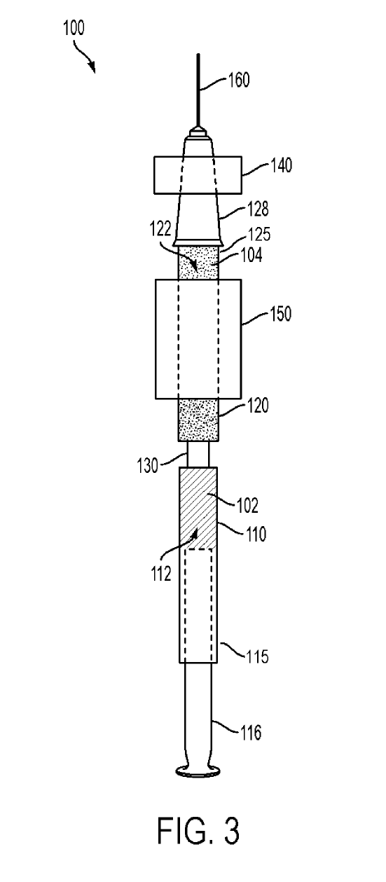

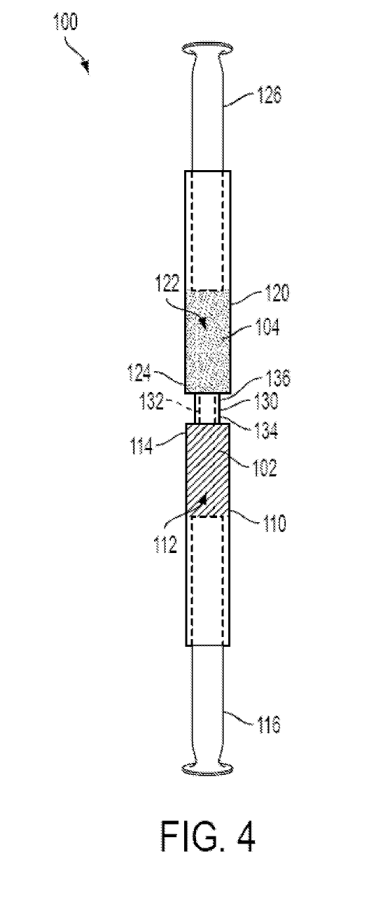

In additional exemplary aspects, and as shown in FIGS. 3-4, a system 100 for delivery of a microparticle composition is disclosed. In these aspects, the system 100 can comprise a first cartridge 110 having an internal cavity 112 into which a predetermined amount of suspension vehicle 102 is contained, a second cartridge 120 having an internal cavity 122 into which a predetermined amount of microparticles 104 is contained, and a connector 130 configured to selectively place the respective internal cavities of the first and second cartridges into operative communication with each other. In a further aspect, the system 100 is configured to allow for the selective introduction of the suspension vehicle 102 from the first cartridge 110 through the connector 130 and into the internal cavity 122 of the second cartridge 120. In another aspect, the system 100 can comprise a sonic energy-generating means 140 configured to contact at least a portion of one or more of the first cartridge 110, the second cartridge 120, and the connector 130. In this aspect, the sonic energy-generating means 140 can be selectively activated to eliminate blockages within the internal cavity of one or more of the first cartridge 110 and the second cartridge 120. It is further contemplated that the sonic energy-generating means 140 can be selectively activated to eliminate blockages within the connector 130. In yet another aspect, the system 100 can be configured to provide for the mixing of the predetermined amount of microparticles 104 and the predetermined amount of suspension vehicle 102 to form a heterogeneous mixture in which the microparticles are distributed substantially uniformly throughout the suspension vehicle. In this aspect, the formed heterogeneous mixture can provide a predetermined dosing level of microparticles.

In another aspect, and with reference to FIG. 4, the connector 130 can have an internal bore 132 extending between a first end 134 and a second end 136. In this aspect, the connector 130 can be configured to selectively connect to a distal end 114 of the first cartridge 110 and into communication with the internal cavity 112 of the first cartridge and to selectively connect to a distal end 124 of the second cartridge 120 and into communication with the internal cavity 122 of the second cartridge. In a further aspect, it is contemplated that the internal bore 132 of the connector 130 can have minimal volumetric dead space. In various aspects, the volumetric dead space of the internal bore 132 of the connector 130 can be ≦10%, ≦9%, ≦8%, ≦7%, 6%, ≦5%, ≦4%, ≦3%, ≦2%, ≦1%, ≦0.9%, ≦0.8%, ≦0.7%, ≦0.6%, ≦0.5%, ≦0.4%, ≦0.3%, ≦0.2%, ≦0.1%, %, ≦0.09%, ≦0.08%, ≦0.07%, ≦0.06%, ≦0.05%, ≦0.04%, ≦0.03%, ≦0.02%, ≦0.01% of the volumes of the respective internal cavities of the first and second containers.

In yet another aspect, it is contemplated that at least a portion of the internal bore 132 of the connector 130 can have a reduced cross-sectional area. Thus, in this aspect, it is contemplated that at least a portion of the internal bore 132 of the connector 130 can have a venturi type shape to encourage mixing of the microparticles 104 and the suspension vehicle 102. Thus, in this aspect, it is also contemplated that at least a portion of the internal bore 132 of the connector 130 can have a static mixing geometry to encourage mixing of the microparticles 104 and the suspension vehicle 102.

Optionally, the connector 130 can be integrally formed with one of the cartridges 110, 120 and can be configured to be selectively coupled to the other cartridge. For example, the connector 130 can be integrally formed as a portion of the distal end 114 of the first cartridge 110. In this example, it is contemplated that at least a portion of the exterior surface of the connector 130 formed in the distal end 114 of the first cartridge 110 can be configured for a fluid-tight connection, such as, for example and without limitation, a luer-lock connection. In this aspect, it is contemplated that a portion of the distal end 124 of the second cartridge 120 can be complementarily formed to selectively form a fluid-tight seal with a portion of the exterior surface of the connector 130. Of course, it is optionally contemplated that the connector 130 can be integrally formed as a portion of the distal end 124 of the second cartridge 120. In this aspect, it is contemplated that at least a portion of the exterior surface of the connector 130 formed in the distal end 124 of the second cartridge 120 can be configured for a fluid-tight connection, such as, for example and without limitation, a luer-lock connection. It is further contemplated that a portion of the distal end 124 of the second cartridge 120 can be complementarily formed to selectively form a fluid-tight seal with a portion of the exterior surface of the connector 130.

In one aspect, it is contemplated that the first and second cartridges 110, 120 can be individually sealed when the respective predetermined amount of suspension vehicle 102 and predetermined amount of microparticles 104 are placed inside the cartridges. In this aspect, the individually sealed cartridges can be maintained separately during shipping and storage. It is contemplated that the respective first and second cartridges 110, 120 can be unsealed immediately prior to initiating the methodology of the present invention.

In one aspect, the first cartridge 110 can comprise a conventional axially movable plunger 116 that is positioned in a friction fit within the internal cavity 112 of the first cartridge. Optionally, as shown in FIG. 4, the second cartridge 120 can similarly comprise an axially movable plunger 126 positioned in a friction fit within the internal cavity 122 of the second cartridge. In a further aspect, the distal end 114, 124 of at least one of the first and second cartridges 110, 120 can be configured for a fluid-tight connection to a needle 160, such as, for example and without limitation, a luer-lock connection 128. In one exemplary aspect, and without limitation, it is contemplated that at least one of the respective first and second cartridges 110, 120 can comprise a conventional syringe. Optionally, as shown in FIG. 3, a proximal end 115, 125 of at least one of the first and second cartridges 110, 120 can be configured for a fluid-tight connection to a needle 160, such as, for example and without limitation, a luer-lock connection 128.

In a further aspect, it is contemplated that the selective introduction of the suspension vehicle 102 from the first cartridge 110 through the connector 130 and into the internal cavity 122 of the second cartridge 120 can be accomplished via application of an external force to the plunger 116 of the first device 110, such axial activation of the plunger toward the distal end 114 of the first cartridge resulting in the forced propulsion of the suspension vehicle 102 through the connector 130 and into the internal cavity 122 of the second container 120, which comprises microparticles 104.

In an additional aspect, the sonic energy-generating means 140 can be configured to contact the distal end 114, 124 of at least one of the first cartridge 110 and the second cartridge 120. Alternatively, the sonic energy-generating means 140 can be configured to contact a portion of the connector 130. In still a further aspect, the sonic energy-generating means 140 can be mounted thereto the outer surface of one or more of the first cartridge 110, the second cartridge 120, and the connector 130. In this aspect, and as shown in FIG. 3, the sonic energy-generating means 140 can comprise a collar element positioned around a portion of one or more of the first cartridge 110, the second cartridge 120, and the connector 130. In another aspect, the sonic energy-generating means 140 can comprise a sonic energy probe, such as, for example and without limitation: digital probe models S-150, 250, and 450 manufactured by Branson Sonifier; 250 and 400 Watt Sonic Ruptor probes manufactured by Omni International; and a model 3000 ultrasonic homogenizer manufactured by Biologics, Inc.

In another aspect, the sonic energy-generating means 140 can have a desired power output. In this aspect, it is contemplated that the desired power output of the sonic energy-generating means 140 can range from about 1 to about 140 Watts. In yet another aspect, it is contemplated that the desired power output of the sonic energy-generating means 140 can range from about 10 to about 120 Watts. In still another aspect, it is contemplated that the desired power output of the sonic energy-generating means 140 can range from about 20 to about 50 Watts. Thus, in various aspects, it is contemplated that the desired power output of the sonic energy-generating means 140 can be 1, 10, 20, 30, 40, 50, 60, 70, 80, 90, 100, 110, 120, 130, or 140 Watts. It is further contemplated that the desired power output of the sonic energy-generating means 140 can fall within a range derived from any two of the above-listed values. Similarly, it is contemplated that the desired power output of the sonic energy-generating means 140 can be any power output falling between any two of the above-listed values.

In a further aspect, the sonic energy-generating means 140 can be in operative communication with a power source. In one exemplary aspect, the power source can be a rechargeable battery. However, it is contemplated that any other conventional power source can be used to activate the sonic energy-generating means 140.

In another aspect, the system can comprise a housing, such as a housing as depicted in FIG. 2. In this aspect, it is contemplated that the first cartridge 110, the second cartridge 120, and the connector 130 can be secured within at least a portion of the housing. In an additional aspect, the sonic energy-generating means 140 can be incorporated into the housing such that sonic energy-generating means is positioned proximate one or more of the distal end 114 of at the first cartridge 110, the distal end 124 of the second cartridge 120, the proximal end 125 of the second cartridge, and the connector 130. Alternatively, the sonic energy-generating means 140 can be secured within the housing such that the sonic energy-generating means is positioned proximate one or more of the distal end 114 of at the first cartridge 110, the distal end 124 of the second cartridge 120, the proximal end 125 of the second cartridge, and the connector 130. In one aspect, the housing can be configured to receive the power source of the sonic energy-generating means 140. In another aspect, the power source of the sonic energy-generating means 140 can be positioned external to the housing. In this aspect, it is contemplated that the power source can be mounted thereto an outer portion of the housing. In a further aspect, the power source of the sonic energy-generating means 140 can be incorporated into the housing.

It is contemplated that movement of the axially-moveable plungers 116, 126 of the respective cartridges 110, 120 and activation of the sonic energy-generating means 140 can be selectively controllable by a user from outside the housing. For example, in one aspect, the system can comprise a controller having an on-off mechanism, such as, for example and without limitation, an on-off switch or an on-off button, that is selectively moveable between an on position and an off position, wherein the on position corresponds to activation of the sonic energy-generating means 140. In this aspect, the controller can be positioned thereon the housing. In another aspect, the controller can comprise means for adjustably controlling the power output of the sonic energy-generating means 140. In a further aspect, it is contemplated that a proximal portion of the axially moveable plunger 116, 126 of each respective cartridge 110, 120 can be accessible by the user from outside the housing.

In another exemplary aspect, the housing can be shaped to conform to the shape of a user's hand. For example and without limitation, it is contemplated that the housing can be substantially cylindrical. It is further contemplated that the housing can have a substantially pen-like shape. In one aspect, the housing can comprise at least one of an injectable moldable plastic and stainless steel. It is contemplated that the housing can comprise an inner surface and an outer surface that are easily cleanable with conventional cleaning materials.

In operation, it is contemplated that a predetermined dosage of microparticles can be delivered into a subject by: providing the first cartridge with a predetermined amount of suspension vehicle sealed therein the internal cavity of the first cartridge; providing the second cartridge with a predetermined amount of microparticles sealed therein the internal cavity of the second cartridge; placing the internal cavity of the first cartridge in communication with the internal cavity of the second cartridge; introducing the suspension vehicle from the first cartridge into the internal cavity of the second cartridge to form a mixture comprising the microparticles; contacting at least a portion of one or more of the first cartridge and the second cartridge with the sonic energy-generating means; and selectively activating the sonic energy-generating means to eliminate blockages within the internal cavity of one or more of the first cartridge and the second cartridge. In one aspect, it is contemplated that the predetermined amount of microparticles and the predetermined amount of suspension vehicle can be mixed together until a heterogeneous mixture is formed in which the microparticles are distributed substantially uniformly throughout the suspension vehicle. In this aspect, the formed heterogeneous mixture can have a predetermined dosing level of microparticles.

In one aspect, the step of contacting at least a portion of one or more of the first cartridge and the second cartridge with the sonic energy-generating means can comprise contacting the distal end of one or more of the first cartridge and the second cartridge with the sonic energy-generating means. In another aspect, it is contemplated that the step of selectively activating the sonic energy-generating means can comprise selectively generating sonic energy at a power output ranging from about 1 Watt to about 140 Watts. In yet another aspect, it is contemplated that the step of selectively activating the sonic energy-generating means can comprise selectively generating sonic energy at a power output ranging from about 10 Watts to about 120 Watts. In still another aspect, it is contemplated that the step of selectively activating the sonic energy-generating means can comprise selectively generating sonic energy at a power output ranging from about 20 Watts to about 50 Watts. Thus, in various aspects, it is contemplated that the step of selectively activating the sonic energy-generating means can comprise selectively generating sonic energy at a power output of 1, 10, 20, 30, 40, 50, 60, 70, 80, 90, 100, 110, 120, 130, or 140 Watts. In these aspects, it is further contemplated that the desired power output of the sonic energy-generating means can fall within a range derived from any two of the above-listed values. Similarly, it is contemplated that the desired power output of the sonic energy-generating means can be any power output falling between any two of the above-listed values.

In another aspect, the connector 130, with its internal bore 132 that extends between the first and second ends 134, 136, can be provided to facilitate the communication between the internal cavities 112, 122 of the first and second cartridges 110, 120. In one exemplary aspect, the connector 130 can be selectively coupled to the respective first and second cartridges 110, 120 to place the internal bore 132 of the connector 130 into communication with the respective internal cavities 112, 122 of the first and second cartridges. Optionally, as described above, it is contemplated that the internal bore 132 of the connector 130 can have minimal volumetric dead space relative to the respective internal cavities 112, 122 of the first and second cartridges 110, 120 for accuracy of the predetermined dosing level of the formed suspension. It is also contemplated that the internal bore 132 of the connector 130 can have a static mixing geometry suitable for assisting or otherwise encouraging the uniform mixing of the microparticles 104 and the suspension vehicle 102. In another aspect, the method can comprise contacting at least a portion of the connector with the sonic energy-generating means.

In a further aspect, the predetermined amount of microparticles 104 and the predetermined amount of suspension vehicle 102 that are originally packaged within the respective first and second cartridges 110, 120 can be uniformly mixed by vibrationally oscillating the second cartridge until the heterogeneous mixture is formed. In this aspect, as depicted in FIG. 3, it is contemplated that the system 100 can further comprise a vibrational collar 150 that can be operatively mounted to a portion of the second cartridge 120. In a further aspect, the vibrational collar 150 can be configured to be selectively oscillated for a time period and at a frequency sufficient to ensure the formation of the heterogeneous mixture in which the microparticles 104 are distributed substantially uniformly throughout the suspension vehicle 102. It is further contemplated that the system 100 can comprise means for adjustably controlling the duration and frequency of the vibrations applied by the vibrational collar 150.

Optionally, the predetermined amount of microparticles and the predetermined amount of suspension vehicle that are originally packaged within the respective first and second cartridges can be mixed by sequentially passing the suspension between the respective internal cavities of the first and second cartridges and through the connector until the heterogeneous mixture, in which the microparticles are distributed substantially uniformly throughout the suspension, is formed. Thus, in one exemplary aspect, the mixing of the predetermined amount of microparticles and the predetermined amount of suspension vehicle to form the heterogeneous mixture can comprise the sequential and repeated axial actuations of the respective plungers of operably coupled first and second devices to pass the mixture of microparticles and suspension vehicle between the respective internal cavities of the first and second devices until the heterogeneous mixture is formed.

In a further aspect, after the heterogeneous mixture, in which the microparticles are distributed substantially uniformly throughout the suspension vehicle, is formed and mixing is complete, the formed heterogeneous mixture can be positioned in a select one of the first and second cartridges. Subsequently, the formed heterogeneous mixture can be transferred to a separate injection device for delivery to the subject or, optionally, a needle can be selectively coupled to the distal end of the respective first or second cartridge (device, syringe, or the like) that contains the formed suspension, so that the heterogeneous mixture, in which the microparticles are distributed substantially uniformly throughout the suspension vehicle, can be selectively delivered to the patient. In one exemplary aspect, the formed heterogeneous mixture can be delivered to the patient by inserting a distal end of the needle at a desired location within a patient and delivering the heterogeneous mixture therein the patient via actuation of the device. It is contemplated that the sonic energy-generating means can be selectively activated to enable a medical practitioner to use smaller needles than would conventionally be used to inject a given composition into a subject, thereby reducing the level of pain experienced by the subject.

In a broad aspect of the invention, the needle 160 is conventional and can have a proximal end, a proximal end opening, a distal end, a distal end opening, and a lumen extending through the needle. It is contemplated that, in one aspect, the distal end of the needle 160 can be sharpened or otherwise suitable for being introduced into the desired tissue. In one exemplary aspect, the needle 160 can comprise a rigid member, such as a metallic member, that can have a substantially circular cross section. In another exemplary aspect, the needle 160 can be coupled to a luer-lock connection of one of the distal end 114 of the first cartridge 110, the distal end 124 of the second cartridge 120, and the proximal end 125 of the second cartridge. In this aspect, as depicted in FIG. 3, it is contemplated that the sonic energy-generating means 140 can be configured to contact the luer-lock connection 128. It is further contemplated that the sonic energy-generating means 140 can be configured to eliminate any blockages existing proximate the interface between the cartridge and the needle 160 to thereby promote flow of the suspension from the cartridge and into the needle.

In one aspect, the needle 160 can have a desired size for insertion into selected tissue of a subject. It is contemplated that the gauge of the needle can be minimized as appropriate to reduce pain in the subject following injection of the suspension into the selected tissue of the subject. In one non-limiting example, the needle 160 can have a gauge ranging from 24 G to 30 G, including 24 G, 25 G, 26 G, 27 G, 28 G, 29 G, and 30 G. However, it is contemplated that the needle 160 can have any gauge that is suitable for a particular injection into the subject.

In a further aspect, the system can further comprise a position subassembly that is configured to control the path of the needle in a plurality of dimensions relative to a target area of a subject. In one aspect, it is contemplated that the plurality of dimension comprises three dimensions, with the third dimension forming an axis that defines the relative depth of an injection. In another aspect, the system can comprise a gauge that is configured to operably measure the correct angle and depth of insertion or injection, i.e., for ensuring correct positioning of injection from the target tissue.

In operation, once the distal end of the needle is positioned within the target area, e.g., the targeted tissue, the plunger can be depressed to drive or force the formed heterogeneous mixture distally. In one exemplary procedure, as the plunger of the device is moved distally or forward, it pushes a desired amount of the mixture into the target area. Optionally, it is contemplated that, in this aspect, once that distal end of the needle is positioned in the desired location, the needle can be removed proximally while concurrently allowing the heterogeneous mixture to remain within the target area.

In exemplary aspects, the system and methods disclosed herein can be used to treat or prevent age related macular degeneration, as well as diseases, illnesses, or conditions relating to retinal edema and retinal neovascularization, including, for example and without limitation, increased or abnormal macular angiogenesis.

In one exemplary aspect, the dosage of the injected material can be a single injection each 3 to 12 months, such as, in various aspects, a single injection about every 3, 6, 9 or 12 months.

In various aspects, the system and methods described herein can be practiced or provided to treat an anterior ocular condition and/or a posterior ocular condition. For example and without limitation, the system and methods can be practiced or provided to treat a condition of the posterior segment of a mammalian eye, such as a condition selected from the group consisting of macular edema, dry and wet macular degeneration, choroidal neovascularization, diabetic retinopathy, acute macular neuroretinopathy, central serous chorioretinopathy, cystoid macular edema, and diabetic macular edema, uveitis, retinitis, choroiditis, acute multifocal placoid pigment epitheliopathy, Behcet's disease, birdshot retinochoroidopathy, syphilis, lyme, tuberculosis, toxoplasmosis, intermediate uveitis (pars planitis), multifocal choroiditis, multiple evanescent white dot syndrome (mewds), ocular sarcoidosis, posterior scleritis, serpiginous choroiditis, subretinal fibrosis and uveitis syndrome, Vogt-Koyanagi- and Harada syndrome; retinal arterial occlusive disease, anterior uveitis, retinal vein occlusion, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemiretinal vein occlusion, papillophlebitis, central retinal artery occlusion, branch retinal artery occlusion, carotid artery disease (CAD), frosted branch angiitis, sickle cell retinopathy, angioid streaks, familial exudative vitreoretinopathy, and Eales disease; traumatic/surgical conditions such as sympathetic ophthalmia, uveitic retinal disease, retinal detachment, trauma, photocoagulation, hypoperfusion during surgery, radiation retinopathy, and bone marrow transplant retinopathy; proliferative vitreal retinopathy and epiretinal membranes, and proliferative diabetic retinopathy; infectious disorders such as ocular histoplasmosis, ocular toxocariasis, presumed ocular histoplasmosis syndrome (POHS), endophthalmitis, toxoplasmosis, retinal diseases associated with HIV infection, choroidal disease associated with HIV infection, uveitic disease associated with HIV infection, viral retinitis, acute retinal necrosis, progressive outer retinal necrosis, fungal retinal diseases, ocular syphilis, ocular tuberculosis, diffuse unilateral subacute neuroretinitis, and myiasis; genetic disorders such as retinitis pigmentosa, systemic disorders with associated retinal dystrophies, congenital stationary night blindness, cone dystrophies, Stargardt's disease and fundus flavimaculatus, Best's disease, pattern dystrophy of the retinal pigmented epithelium, X-linked retinoschisis, Sorsby's fundus dystrophy, benign concentric maculopathy, Bietti's crystalline dystrophy, and pseudoxanthoma elasticum; retinal tears/holes such as retinal detachment, macular hole, and giant retinal tear; tumors such as retinal disease associated with tumors, congenital hypertrophy of the retinal pigmented epithelium, posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, and intraocular lymphoid tumors; punctate inner choroidopathy, acute posterior multifocal placoid pigment epitheliopathy, myopic retinal degeneration, acute retinal pigment epithelitis, retinitis pigmentosa, proliferative vitreal retinopathy (PVR), age-related macular degeneration (ARMD), diabetic retinopathy, diabetic macular edema, retinal detachment, retinal tear, uveitus, cytomegalovirus retinitis and glaucoma and conditions involving ocular degeneration, such as neurodegeneration of retinal ganglion cells.

In various aspects, the compositions or heterogeneous mixtures used for the disclosed methods can have from about 1 to 500 mg, 50 to 400 mg, 50 to 300 mg, 50 to 200 mg, 50 to 150 mg, or about 100 mg of microparticles substantially uniformly suspended in the suspension vehicle. The compositions or heterogeneous mixtures, in one aspect, can comprise from about 1% to about 50% solids and in another aspect from about 10% to 40% solids and in another aspect from about 20% to about 30% solids. In one example, and without limitation, the compositions or heterogeneous mixtures used for the disclosed methods can have from about 10 mg to about 150 mg of microparticles substantially uniformly suspended in the heterogeneous mixture, wherein the heterogeneous mixture comprises from about 20% to about 30% solids. As described above, for an exemplary eye procedure, the microparticles and compositions disclosed herein can be delivered by injecting them intravitrealy at 10 to 150 μL total volume per injection using a needle, such as, for example and without limitation, a 25-G UTW needle.

In one aspect, the microparticles that can be used in the disclosed methods can have an average or mean particle size ranging from about 5 μm to about 125 μm. In another aspect, the range of mean particle size can be from about 20 μm to about 90 μm. In yet another aspect, the range of mean particle sizes can be from about 50 μm to about 80 μm. In an additional aspect, the nanoparticles that can be used in the disclosed methods can have an average or mean particle size ranging from about <1 nm to about 1000 nm. In a further aspect, the range of mean particle size can be from about 50 μm to about 600 μm. In still a further aspect, the range of mean particle sizes can be from about 100 μm to about 300 μm. It is contemplated that the particle size distributions can be measured by laser diffraction techniques known to those of skill in the art.

In one aspect, a drug product can be used to prepare the disclosed microparticles. In this aspect, the drug product that is used during preparation of the microparticles can comprise one or more water soluble carriers or excipients. Such carriers or excipients can generally include sugars, saccharides, polysaccharides, amino acids, surfactants, buffer salts, bulking agents, and the like. A non-limiting example of an excipient is 2-(hydroxyl-methyl)-6-[3,4,5-trihydroxy-6-(hydroxymethyl)tetrahydropyran-2-yl]oxy-tetrahydropyran-3,4,5-triol, “trehalose.” One aspect of the disclosed process includes a bulk drug product used during preparation of the microparticles comprising about 1 wt % to about 50 wt % trehalose based on the weight of drug in the starting bulk drug product. In a further aspect, the bulk drug product used during preparation of the microparticles can comprise about 10 wt. % to about 50 wt. % trehalose based on the weight of drug in the starting bulk drug product. In a non-limiting example of this aspect the bulk drug product can comprise about 25 wt % to about 35 wt % trehalose. Another non-limiting example of an excipient is the surfactant polysorbate 20 (or Tween 20). One optional aspect of the disclosed process includes a bulk drug product used during preparation of the microparticles comprising about 0.01 wt % to about 5 wt % polysorbate 20 based on the weight of drug in the starting bulk drug product. In yet a another aspect, the bulk drug product used during preparation of the microparticles can comprise about 0.05 wt % to about 0.25 wt % polysorbate 20 based on the weight of drug in the starting bulk drug product. In a non-limiting example of this aspect, the bulk drug product can comprise about 0.1 wt % polysorbate 20. In further aspects, the bulk drug product can contain two or more such carriers or excipients. A non-limiting example includes a bulk drug product comprising about 25 wt % to about 35 wt % trehalose and about 0.1 wt % polysorbate 20 based on the weight of drug in the starting bulk drug product.

The polymers used as the microparticle matrix material can be a single homopolymer, for example, poly(D,L-lactide), or a blend of two or more homopolymers and/or copolymers. When two or more polymers comprise the matrix material, the formulator can use any of a variety of methods known to those skilled in the art. A non-limiting example of the blending of two polymers includes the following procedure: charging the desired amount of the polymers to a suitable vessel containing an amount of one or more organic solvents; sealing the vessel, such as, for example, by stoppering the vessel; agitating the contents of the vessel until the polymer is completely dissolved or dispersed; and storing (or directly using) the dispersed phase of the mixture to form a primary emulsion for the disclosed process.

### EXAMPLES

The following examples are put forth so as to provide those of ordinary skill in the art with a complete disclosure and description of how the compounds, compositions, articles, systems, and/or methods described and claimed herein are made and evaluated, and is intended to be purely exemplary and is not intended to limit the scope of what the inventors regard as their invention.

The following examples were practiced using a syringe comprising a barrel in communication with a needle. The barrel contained a composition for injection. As used in the following examples, an injection “failed” when one or more blockages occurred within the barrel, thereby leading to inadequate flow of the composition from the barrel into and through a needle. As used in the following examples, an injection “passed” or “succeeded” when the composition freely flowed from the barrel into and through the needle.

### Example 1

A simulated injection with microsphere suspensions was performed. The microsphere suspension was 65:35 DL-lactide-co-glycolide (DLG). During some of the simulated injections, a 25 gauge needle was contacted with a Branson Sonifier 450 micro-sonic probe to deliver a low level of sonic energy (corresponding to Setting 1, the lowest setting on the probe). Results of the simulated injections are shown in Table 1. As described in Table 1, the injections that were conducted without application of sonic energy failed, while the injections that were conducted with application of sonic energy succeeded.

TABLE 1

| | Percent | Needle | Sonic Probe | |
|---|---|---|---|---|
| Description | Solids | Gauge | Settings | Pass/Fail |
| 65:35 PLG | 20 | 25 g | None | Fail |
| 65:35 PLG | 20 | 25 g | Setting 1 - | Pass |
| | | | 10% Pulse | |
| 65:35 PLG | 50 | 25 g | None | Fail |
| 65:35 PLG | 50 | 25 g | Setting 1 - | Pass |
| | | | 10% Pulse | |

### Example 2

A placebo (empty) microparticle formulation was prepared using an emulsion-based process. A 65:35 DL-lactide-co-glycolide (DLG) was obtained from Lakeshore Biomaterials. Ethyl Acetate (Fisherbrand Optima, ACS grade) was used as received from Fisher Scientific. Poly (vinyl alcohol) (PVA), ultra pure grade (87.5-89% hydrolysis) was purchased from Amresco (Solon, Ohio). The emulsion-based process used was a solution continuous process. A 20 wt % dispersed phase (DP) was prepared by dissolving 30 grams of polymer in 120 grams of ethyl acetate. A continuous phase (CP) solution was prepared by saturating 1000 grams of 2 wt % PVA with 82 grams of ethyl acetate. To prepare the microparticle formulation, a Silverson L4R-T mixer with a laboratory in-line mixer head with a general-purpose disintegrating head (stator screen) was configured. The dispersed phase (DP) solution and the continuous phase (CP) solution were delivered separately into the inlet assembly of the mixer head. The DP and CP solutions were delivered into the mixer head at flow rates of 20 g/min and 125 g/min, respectively. A mixer stir speed of 1200 rpm was selected. The effluent emulsion from the mixer was immediately diluted with additional water (the external phase or EP solution) at an emulsion to EP ratio of approximately 1:15. All of the diluted effluent emulsion was collected in a tank. The tank's contents were mixed for 2 hours before collection on a set of 125 and 25 micron test sieves. The material collected on the 25 micron sieves was further rinsed with water. The rinsed microparticles were dried by lyophilization. The dried formulation was sieved again in a dry state through a 125 micron test sieve to remove any agglomerates that could have formed during drying.

The injection vehicle used in the syringability (injectability) studies was composed of 0.5 wt % sodium carboxymethyl cellulose (CMC) and 0.1 wt % Tween 80 (Polysorbate 80). Becton Dickinson (BD) 1-mL BD Luer-Lok™ syringes (BD Product Number 309628) were used for the syringability studies also. Syringe needles used were Becton-Dickinson BD precision glide needles of standard wall thickness. Syringe needle product numbers are listed in Table 2.

TABLE 2

| Gauge | Length | | BD Product Number |
|---|---|---|---|
| 20 G | 1 | inch | 305175 |
| 25 G | ⅝ | inch | 305122 |

Triplicate samples of a microparticle formulation were prepared for syringability testing. A predetermined amount of the microparticle formulation was weighed into a syringe based on the suspension concentration (percent solids) listed in Table 3.

TABLE 3

| Percent solids | Amount of microparticles | Injection vehicle |
|---|---|---|
| to be tested | weighed into Syringe 1 | used in Syringe 2 |
| 20% | 100 mg | 0.4 cc |
| 50% | 200 mg | 0.2 cc |

Using a syringe connector, a syringe containing a predetermined amount of injection vehicle is attached to the syringe containing the weighed microparticles. The contents of the two syringes were mixed back and forth for approximately 30 passes.

Immediately after mixing, the suspension was expelled out of the syringe through the syringe needle into a vial. The injectability of an individual syringe (or trial) was considered a “pass” if the complete contents of the syringe were expelled without any clogs or blockages that stopped the flow of the suspension out of the needle or if there was no noticeable change in pressure that interrupted the constant, steady depression of the plunger by the operator. For a set of triplicate samples, all individual samples must pass for the testing condition to be considered successful.

Triplicate test samples were prepared for each set of conditions listed in Table 4. Sonic energy was not applied during the injection of any of these samples.

TABLE 4

| Percent solids | Syringe needle | Injectability results |
|---|---|---|
| to be tested | gauge (g) size | (Pass/fail) |
| 20% | 20 g | pass |
| 20% | 25 g | fail |
| 50% | 25 g | fail |

A Branson sonifier model S-450A (Branson Ultrasonic's Corporation, USA) was set up with a tapered micro-tip probe (⅛ inch, part no. 101-148-062). The instrument was set with an amplitude setting of 1 (10%) and a duty cycle of 10% pulse. The output of the sonifier was evaluated at different amplitude settings while keeping the duty cycle setting as shown above constant along with type of probe. A 25 gauge needle was attached to a 1 mL empty syringe. The tip of sonifier probe was touched to the luer hub of the needle and output was recorded off the instrument output readout. Output recordings are listed in Table 5. The use of the microtip probe increased the measured output by a multiple of 3.5 times. Because the sonifier probe was not immersed in a fluid during the measurement, the output measurement was lower than referenced as the amplitude setting was increased. A calculated range of output is shown.

TABLE 5

| Amplitude | Referenced | Measured % | Calculated |
|---|---|---|---|
| setting | Output, Watts | Output | output, watts |
| 1 | 70 | 10 | 20-70 |
| 3 | 210 | 15 | 30-105 |
| 7 | 490 | 20 | 40-140 |

Triplicate test samples were prepared for each of the conditions listed in Table 6. After preparation of the suspension and prior to pressing the plunger to perform the simulated injection, the sonifier was setup and tip was touched to the luer hub of the needle. The plunger was depressed and injection performed while maintaining the sonic energy that was applied to the needle hub.

TABLE 6

| Percent solids | Syringe needle | Injectability results |
|---|---|---|
| to be tested | gauge size | (Pass/fail) |
| 20% | 25 g | Pass |
| 50% | 25 g | Pass |

A application of sonic energy showed a enhanced injectability compared to no application of sonic energy. The use of sonic energy showed no dramatic effect upon the average particle size of the microparticles. Successful (pass) collected suspensions from test conditions 1 and 2 were evaluated for particle size using a Coulter LS particle size analyzer. Results are provided in Table 7.

TABLE 7

| Microparticle | Percent | Needle | Sonic | Measured | Mean size, |
|---|---|---|---|---|---|
| Formulation, # | solids, % | size, G | energy | % output | microns |
| 1 | 20 | 20 | no | NA | 41.7 |
| 1 | 20 | 25 | yes | 10 | 40.6 |

The amount of sonic energy that could be used on a microparticle suspension but not have a dramatic effect upon particle size was also evaluated. A second placebo microparticle formulation was prepared as described above. For this formulation, a 75:25 DLG polymer was used instead of a 65:35 DLG. The 75:25 DLG polymer was obtained from Lakeshore biomaterials. Using conditions described in test conditions 1 and 2, successful (pass) collected suspensions were evaluated for particle size using a Coulter LS particle size analyzer. Results are summarized in Table 8.

TABLE 8

| Micro- | | | | | | |
|---|---|---|---|---|---|---|
| particle | Percent | | | | | Mean |
| Formulation, | solids, | Needle | Sonic | Sonifier | Measured | size, |
| # | % | size, G | energy | setting | % output | microns |
| 2 | 20 | 20 | no | NA | NA | 65.8 |
| 2 | 20 | 25 | yes | 1 | 10 | 62.2 |
| 2 | 20 | 25 | yes | 3 | 15 | 67.9 |
| 2 | 20 | 25 | yes | 7 | 20 | 67.5 |

The mean particle size for the formulation suspended in an injection vehicle with no exposure to a syringe or needle was determined to be 66.2 microns. At a setting 7, which was the maximum setting for the sonifier tip listed by the manufacturer, the use of sonic energy did not dramatically affect the particle size of the formulation.

1. A system for delivery of a suspension, comprising:
a needle;
a cartridge having an internal cavity into which a predetermined amount of the suspension is sealed therein, the cartridge having an outer surface and a distal end configured for fluid-tight connection to the needle;
an axially movable plunger positioned in a friction fit within the internal cavity of the cartridge; and
sonic energy-generating means configured to contact at least a portion of the outer surface of the cartridge,
wherein, during movement of the axially movable plunger, the sonic energy-generating means can be selectively activated to eliminate blockages within the internal cavity of the cartridge.

2. The system of claim 1, wherein the needle has a gauge ranging from 24 G to 30 G.

3. They system of claim 1, wherein the sonic energy-generating means is configured to contact the distal end of the cartridge.

4. The system of claim 3, wherein the distal end of the cartridge has a luer-lock-configuration.

5. The system of claim 3, wherein the sonic energy-generating means is mountable thereto the outer surface of the cartridge.

6. The system of claim 1, wherein the suspension comprises microparticles.

7. The system of claim 1, wherein the sonic energy-generating means comprises a sonic energy probe.

8. The system of claim 1, further comprising a housing, wherein the needle, the cartridge, the axially-movable plunger, and the sonic energy-generating means are secured within the housing such that the sonic energy-generating means is proximate the distal end of the cartridge.

9. The system of claim 8, wherein movement of the axially-movable plunger and activation of the sonic energy-generating means are selectively controllable by a user from outside the housing.

10. The system of claim 1, wherein the sonic energy-generating means is configured to generate sonic energy at a power output ranging from about 1 Watt to about 140 Watts.

11. The system of claim 1, wherein the sonic energy-generating means is configured to generate sonic energy at a power output ranging from about 10 Watts to about 120 Watts.

12. The system of claim 1, wherein the sonic energy-generating means is configured to generate sonic energy at a power output ranging from about 20 Watts to about 50 Watts.

13. A system for delivery of a microparticle composition, comprising:
a first cartridge having an internal cavity into which a predetermined amount of a suspension vehicle is sealed therein;
a second cartridge having an internal cavity into which a predetermined amount of microparticles is sealed therein;
a connector having an internal bore extending between a first end and a second end, the connector configured to selectively connect to a distal end of the first cartridge and into communication with the internal cavity of the first cartridge and to selectively connect to a distal end of the second cartridge and into communication with the internal cavity of the second cartridge;
means for introducing the suspension vehicle from the first cartridge through the connector and into the internal cavity of the second cartridge; and
means for substantially uniformly mixing the predetermined amount of microparticles and the predetermined amount of suspension vehicle to form a heterogeneous mixture in which the microparticles are distributed substantially uniformly throughout the suspension vehicle, wherein the heterogeneous mixture has a predetermined dosing level of microparticles.

14. The system of claim 13, further comprising a sonic energy-generating means configured to contact at least a portion of one or more of the first cartridge, the second cartridge, and the connector, wherein the sonic energy-generating means can be selectively activated to eliminate blockages within one or more of the internal cavity of the first cartridge, the internal cavity of the second cartridge, and the connector.

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. A method for delivering a suspension, comprising:
providing a cartridge having an internal cavity into which a predetermined amount of the suspension is sealed therein, the cartridge having an outer surface and a distal end configured for fluid-tight connection to a needle;
selectively axially moving a plunger positioned in a friction fit within the internal cavity of the cartridge;
contacting at least a portion of the outer surface of the cartridge with sonic energy-generating means; and
selectively activating the sonic energy-generating means to eliminate blockages within the internal cavity of the cartridge.

39. The method of claim 38, wherein the needle has a gauge ranging from 24 g to 30 g.

40. They method of claim 38, wherein the step of contacting at least a portion of the outer surface of the cartridge with sonic energy-generating means comprises contacting the distal end of the cartridge with the sonic energy-generating means.

41. The method of claim 40, wherein the distal end of the cartridge has a luer-lock-configuration.

42. The method of claim 41, wherein the sonic energy-generating means is mounted thereto the outer surface of the cartridge.

43. The method of claim 38, wherein the suspension comprises microparticles.

44. The method of claim 38, wherein the sonic energy-generating means comprises a sonic energy probe.

45. The method of claim 38, wherein the step of selectively activating the sonic energy-generating means comprises selectively generating sonic energy at a power output ranging from about 1 Watt to about 140 Watts.

46. The method of claim 38, wherein the step of selectively activating the sonic energy-generating means comprises selectively generating sonic energy at a power output ranging from about 10 Watts to about 120 Watts.

47. The method of claim 38, wherein the step of selectively activating the sonic energy-generating means comprises selectively generating sonic energy at a power output ranging from about 20 Watts to about 50 Watts.

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)
